(19) United States
(12) Patent Application Publication
(10) Pub. No.: US 2003/0200115 A1
(43) Pub. Date: Oct. 23, 2003

(54) Method for reporting medical data

(75) Inventor: Arun Arora, Oyster Bay, NY (US)

Correspondence Address: DARBY & DARBY P.C., Post Office Box 5257, New York, NY, 10150-5257, US

(73) Assignee: Arun Arora

(21) Appl. No.: 10/131,778

(22) Filed: Apr. 23, 2002

(51) Int. Cl. G06F 17/60; A61B 5/00

(52) U.S. Cl. 705/2; 600/300

(57) ABSTRACT

The present invention is directed to a method for converting data acquired from medical reports and for converting drug dosage amounts in drug prescriptions to a universal reference range by which all other medical reports and prescriptions can be evaluated and used for diagnosis and treatment in the medical field

### BACKGROUND OF THE INVENTION

1. Field of the Invention

The present invention relates generally to a method of reporting medical information. In particular, the present invention relates to a method for reporting medical information such that health care professionals use a universal reference range to evaluate medical reports and to determine drug dosage disbursal amounts.

2. Description of Related Art

Life expectancy for humans rose from 47 years in 1901 to 80 years in the 20^{th} century. Studies indicate that the fastest growing population include people 85 years and older. With the rapid increase of the elderly population, the medical profession faces new challenges in the 21^{st} century.

A doctor in 1901 could carry all their medications and instruments in a single bag. The same early 20^{th} century doctor diagnosed and treated a patient exclusively on the patient's medical history and physical exam. While patient history and physical examination remain in part the basis of evaluation today, no patient diagnosis or evaluation would be complete without lab tests, imaging reports, and interventional technology.

In addition to the technological advancements made in medicine, numerous specialties and sub-specialties have evolved. The advent of these numerous specialties and sub-specialties demonstrate that today, no one doctor can evaluate each available lab test, image report, and drug. While these technological advancements enable a more educated diagnosis and treatment of a patient's condition, at the same time these technological advancements further distance any single physician from knowing of all the best tests and drugs available on the market.

Today's health care professionals, whether a primary care physician, specialist, nurse practitioner or physician's assistant, face the challenge of interpreting each lab test, imaging report, and medication dosage amount each time technology advances. Many common diseases rely exclusively on lab tests for diagnosis and therapeutic monitoring. Accurate and efficient interpretation of lab results is especially important for these common diseases.

In addition to the advances in technology, the world has becomes increasingly globalized. Often times the same test administered in a different environment with a different machine and a different chemical agent produces different test results. Each test accompanying these different environments, machines and chemical agents produce different reference ranges and ways of interpreting the test results as the test results relate to the different reference ranges. Therefore, not only must a health care provider be cognizant of the various tests and medications available to diagnosis and treat but also that various reference ranges apply to those tests and medications.

By way of example, every time a lab sends a test report, the patient and the healthcare provider evaluating the test must determine:

(1) What is the reference range for the test conducted?

(2) What were the test results reported last time the test was conducted?

(3) What is the trend analysis over the last few test reports?

(4) Was the same technology used in all previous test reports?

(5) Was the same reference range used in all previous test reports?

In addition, each time a prescription is prescribed, the patient and the health care provider must determine:

(1) What is the appropriate drug dosage reference range for this particular drug?

(2) What was the drug dosage prescribed last time?

(3) What is the trend analysis for this drug dosage?

And if a new drug is prescribed, the health care provider must understand the new drug dosage reference ranges and run through the same three-step test. If the health care provider prescribes a new drug and accidently uses the old drug's reference range, an overdose can occur.

Last decade saw a new challenge in medical care with the evolution of Managed care and HMO's. As a result, health care professionals have increased burdens and pressures on their time. These increased burdens and pressures make error more likely.

Consequently, what is needed is a simple method of reporting test results in lab reports and reporting drug prescriptions so that patients and the health care providers can optimally use lab reports and prescriptions for diagnosis and treatment.

### SUMMARY OF THE INVENTION

The present invention is a method for converting data from data proportionately related to a reference range particular to a test to data proportionately related to a universal reference range common to a plurality of tests. First data is acquired data from a test. The test has a reference range particular to that test. This reference range has a low value and a high value. The acquired data proportionately relates to the first reference range. The proportionate relationship of the acquired data to the first reference range indicates a health condition. Then the acquired data is converted to another reference range. The second reference range is universal to a plurality of tests. The second reference range also has a low value and a high value. The converted data proportionately relates to the second reference range. The proportionate relationship of the converted data to the second reference range substantially parallels the proportionate relationship of the acquired data to the first reference range. The proportionate relationship of the converted data to the second reference range indicates the same health condition as the proportionate relationship of the acquired data to the first reference range.

The present invention is a method for converting drug prescriptions to a universal reference range common to a plurality of drug prescriptions. First, a prescribed dosage amount for a drug is acquired. The prescribed drug dosage amount proportionately relates to a reference range particular to the prescribed drug. The reference range has a minimum dosage amount and a maximum dosage amount. Then the prescribed dosage amount is converted to another reference range. The other reference range is universal to a plurality of drugs. The second reference range also has a minimum dosage amount and a maximum dosage amount. In addition the proportionate relationship of the converted dosage amount to the second reference range substantially equals the proportionate relationship of the acquired dosage amount to the first reference range.

### BRIEF DESCRIPTION OF THE ILLUSTRATIVE EMBODIMENTS

The foregoing and other features of the present invention will be more readily apparent from the detailed description of the illustrative embodiments wherein like reference numbers refer to similar elements throughout several views and in which;

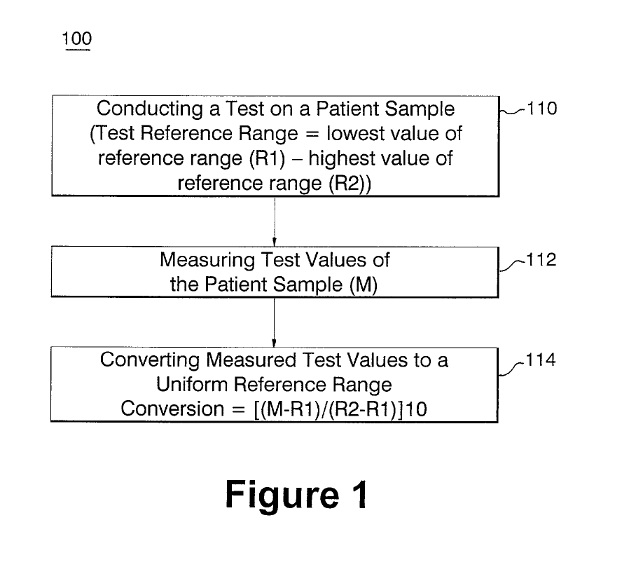

FIG. 1 depicts a flowchart depicting a conversion method in accordance with the present invention;

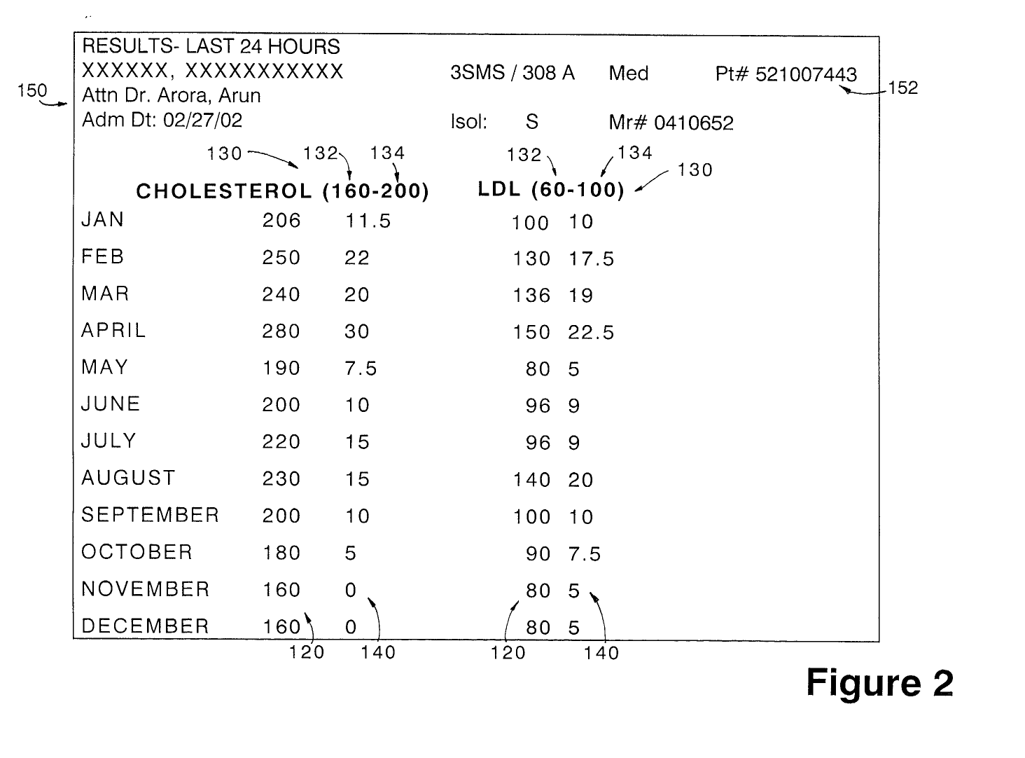

FIG. 2 depicts a table demonstrating the conversion method of FIG. 1 in accordance with a first illustrative embodiment;

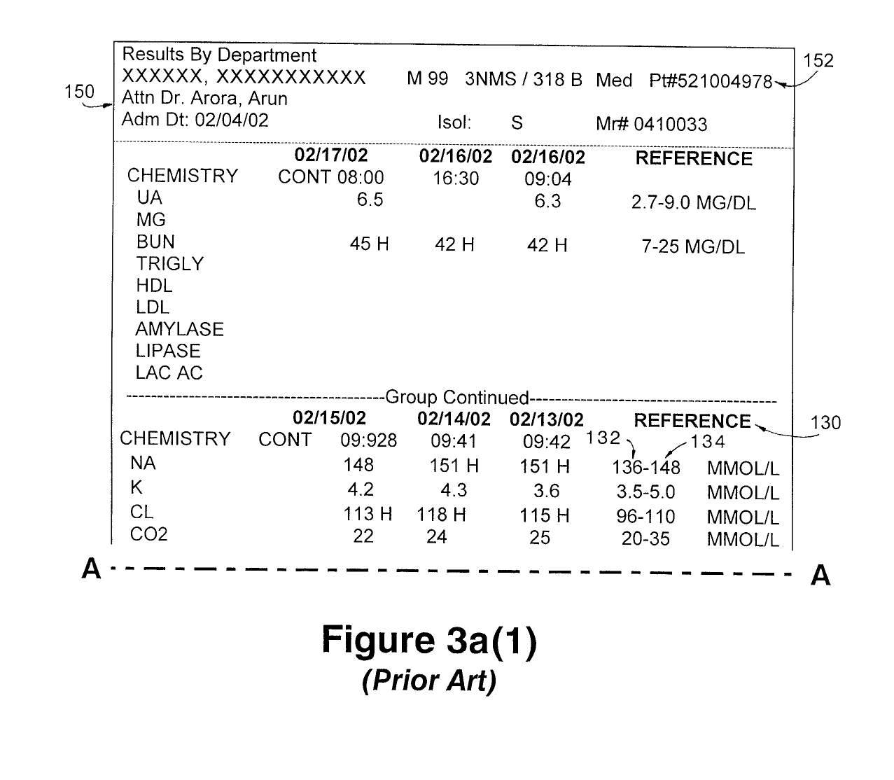

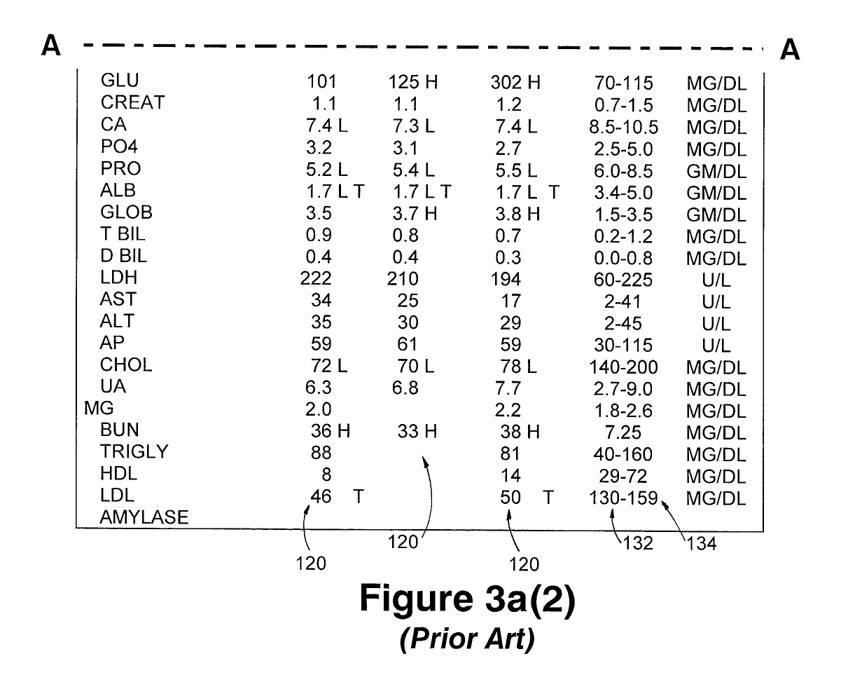

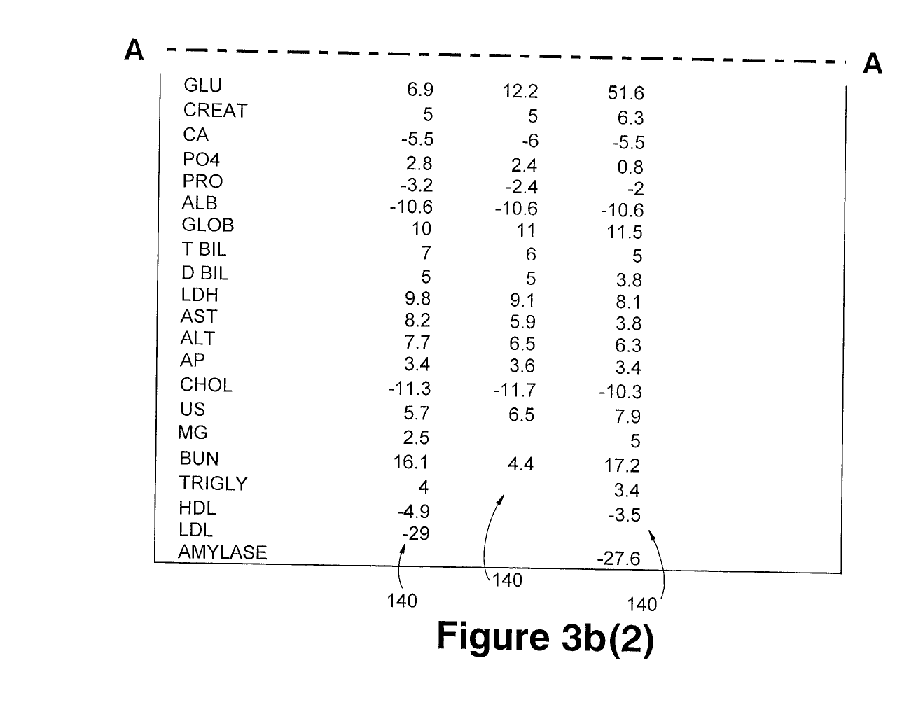

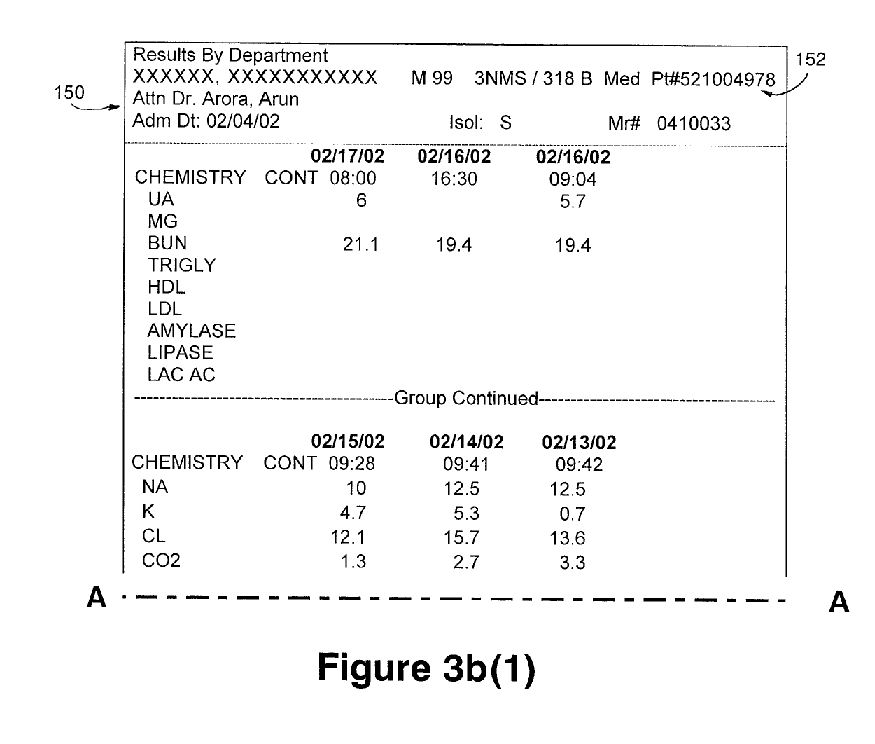

FIG. 3a depicts a prior art blood chemistry report;

FIG. 3b depicts the blood chemistry report of FIG. 3a as modified in accordance with the present invention;

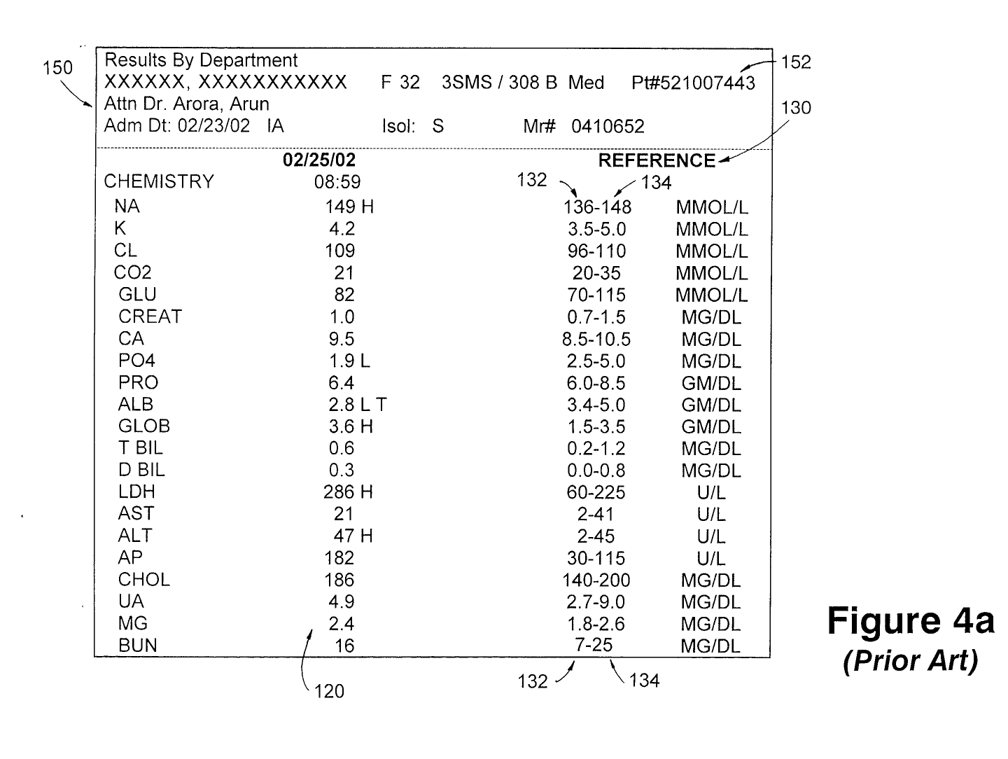

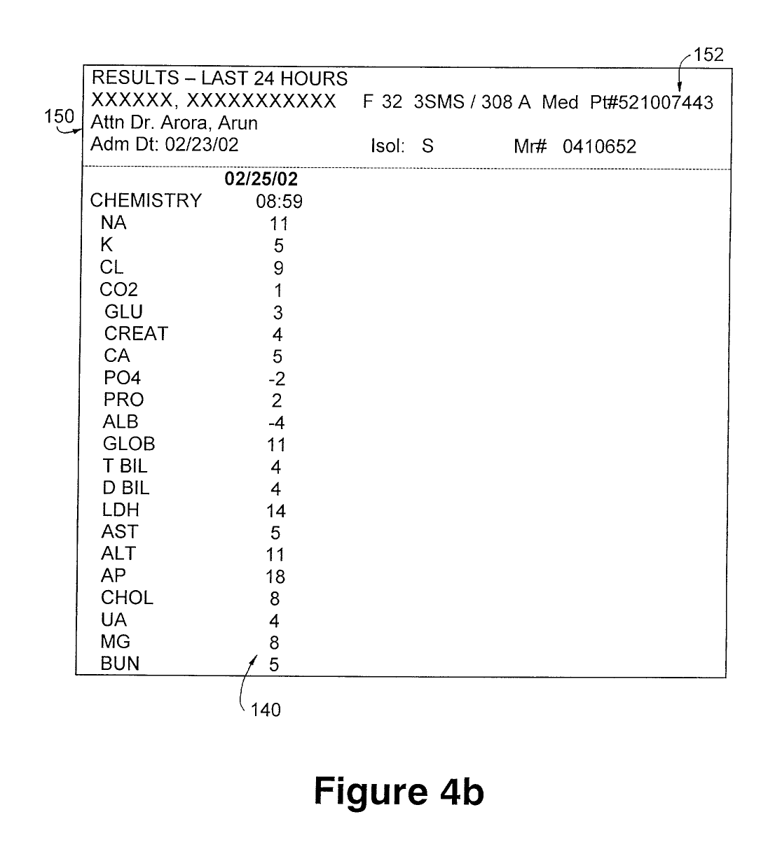

FIG. 4a depicts a further prior art blood chemistry report;

FIG. 4b depicts the blood chemistry report of FIG. 4a as modified in accordance with the present invention;

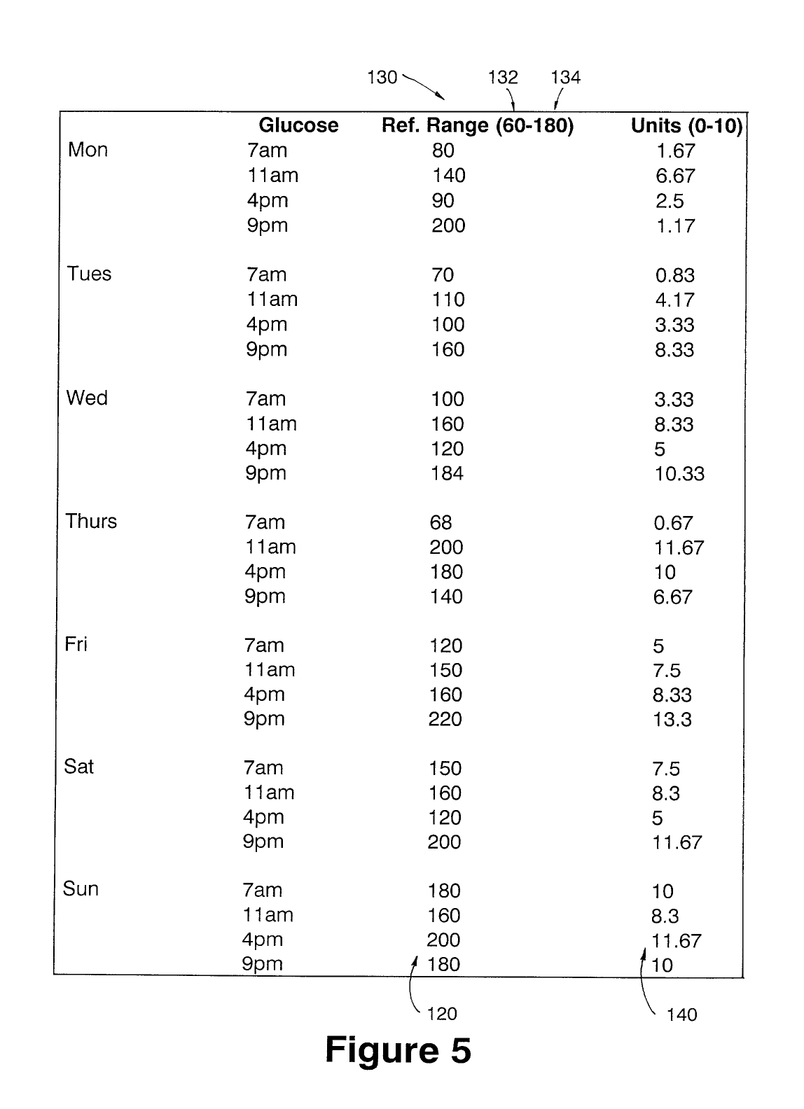

FIG. 5 depicts a further table demonstrating the conversion method of FIG. 1; and

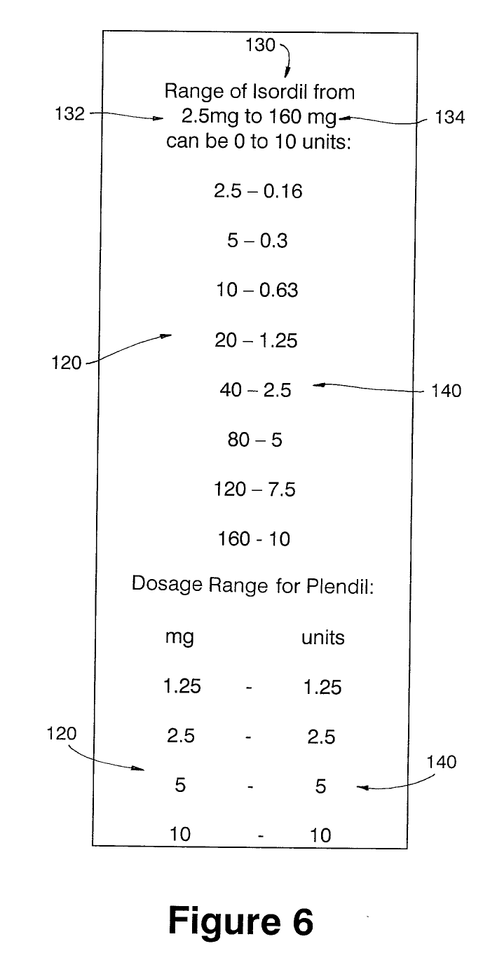

FIG. 6 depicts the conversion process of the present invention in accordance with a second illustrative embodiment.

### DETAILED DESCRIPTION OF THE ILLUSTRATIVE EMBODIMENTS

By way of example, the illustrative embodiments of the present invention depict a conversion method for evaluating test data in the medical field. In medicine, rarely can a single value within a single test report convey conclusively the condition of a person's health. Instead, tests are reported with a range of acceptable values, or a reference range. Health care professionals evaluate, diagnose and treat patients based in part on how a patient's test results compare with the test's reference range.

Often times, when a person's health fails, a health care professional will take a sample of that person's bodily fluid, e.g. blood or urine. The sample is then sent to a lab for testing. The health care professional uses the patient's physical examination to determine which tests the health care professional should instruct the lab to conduct. In turn, the health care professional uses the tests to diagnose and treat.

When the lab report returns to the health care professional, the lab report returns with a test result and a set of reference ranges particular to the test. Generally speaking, the reference range indicates how a healthy person would evaluate under the test. Because lab equipment varies, the reference range for one lab does not always coincide with the reference range for the same test at a different lab or even the reference range for the same test with different equipment at the same lab. In addition, each test has its own reference range. Consequently, when the health care professional uses the lab report for diagnosis and treatment, the health care professional must be cognizant of all the applicable reference ranges. One lab report could have upwards of twenty test results with a set of twenty reference ranges accompanying those twenty test results.

The present invention simplifies the reference range dilemma. The present invention converts the various reference ranges into one common reference range. All tests conducted by all equipment at all laboratories are converted to one common reference range. Consequently, rather than requiring a health care professional to be cognizant of twenty test results with twenty reference ranges, the health care professional must only be cognizant of twenty test results with one common reference range.

FIG. 1 depicts a method for converting reference ranges associated with lab equipment of various tests conducted in various laboratories into a common reference range in accordance with the present embodiment. First, a test is conducted on a patient to evaluate a patient's health (step 110). The test can include, but is not limited, taking samples from the patient. The samples can include, but are not limited to blood, urine and tissue samples. For each test conducted, the test has a reference range determinative of a patient's health. As discussed above, rarely can a single test value determine a patient's health. Instead, health care professionals speak in a language of reference ranges.

Each reference range has a high and low value, R2 and R1 respectively. Generally speaking, the reference range anticipates a healthy reference range for a healthy patient. Therefore, should a patient's test results fall within the reference range, the patient's test result indicates a healthy condition. However, it is equally contemplated and within the scope of the invention that the reference range indicate a range other than that of a healthy adult. For example, the reference range could indicate that should the patient fall within the range's boundaries that an unhealthy condition exists.

Once the test has been conducted, (step 110), data values are measured from the patient's test samples (step 112). The acquired value (step 112) and its proximity to the reference range of the test conducted (step 110) indicate the existence or nonexistence of a health condition to health care professionals evaluating the test results. However, as discussed above, various laboratories use various pieces of equipment which leads to various reference ranges for the same test. Therefore, once the test has been conducted (step 110) and data values have been measured (step 112), the acquired values are converted to a common reference range to be utilized by all laboratories for all tests conducted regardless of the equipment (step 114) conducting the test. The conversion equation follows:

Converted Test Values=[(M−R1)/(R2−R1)]10 (Equation 1)

Once the measured test values have been converted (step 114), every test report lists the acquired values as converted by equation 1. Equation 1 converts the measured test values from a first reference range to a common reference range. The acquired data values proportionately relate to the first reference range. Equation 1 maintains the same proportionate relationship with the common reference range. The common reference range of equation 1 has a low value of 0 and a high value of 10. The acquired test values are converted to fit the 0-10 common reference range.

Equation 1 retains proportionally the acquired values as they related to the original reference range. If an acquired value tested higher than the original reference range, the conversion formula of equation 1 yields a converted test value higher than 10. It should be understood that while equation 1 converts the acquired value to a common reference range of 0-10 other values can substitute for the common reference range as long as the test reports conform to a common reference range. For example, other common reference ranges could 0-100 or 0-1000.

FIGS. 2-6 further illustrate the method of the present invention. FIGS. 2-5 depict various blood chemistry reports. FIGS. 2 and 5 compare the prior art reports with the blood work reports in accordance with the method of the present invention. FIGS. 3a and 4a depict prior art blood work reports and FIG. 3b and FIG. 4b depict blood work reports in accordance with the conversion method of the present invention. FIG. 6 depicts how the conversion method of the present invention can be utilized for medications as well as blood chemistry reports.

FIG. 2 depicts a table demonstrating the conversion method of FIG. 1 in accordance with a first illustrative embodiment. The blood chemistry of the patient 152 was checked once each month from January until December. In this particular, the patient's cholesterol and LDL levels were measured. As shown in FIG. 2, for the cholesterol tests conducted on this particular piece of lab equipment the reference range 130 is “160-200.” While in the lab report of FIG. 2, the cholesterol reference range is “160-200,” the cholesterol reference range in FIGS. 3a is “140-200.” Different labs use different equipment creating the reference range discrepancy seen in FIGS. 2 and 3a. In addition to cholesterol, the lab report of FIG. 2 depicts the patient's LDL (Low Density Lipoprotein). For this lab report, the LDL reference range 130 was “60-100.”

FIG. 2 depicts two columns for both the Cholesterol and LDL readings. For each test, the left numeric column depicts the acquired or the measured test value (M) as it compares against the original reference range and the right numeric column depicts the converted test value (C) as it compares against the converted common reference range (0-10). In January, the patient had a measured cholesterol value of “206” and a converted cholesterol reading of “11.5.” The measured cholesterol value “206” exceeds the reference range 130 and proportionately, the converted cholesterol reading “11.5” exceeds the common reference range of “0-10.” In June, the cholesterol reading reports an acquired value “200.” Because for June the measured cholesterol value “200” is within the original reference range of “160-200,” consequently the converted value “10” is also within the common reference range of “0-10.” In November, the measured cholesterol value 120 for patient 152 was at its lowest “160” but still within the original reference range “160-200.” Consequently, when the November cholesterol reading is converted, the converted reading “0” is at its lowest but still within the converted reference range of 0-10.

The blood chemistry report of FIG. 2 also depicts an LDL reading of the blood work. The original reference range for the LDL readings was “60-100.” In January, the patient had a measured LDL value that was high “100” but still within the original LDL reference range “60-100.” Consequently, the converted LDL value “10” was high but still within the converted reference range “0-10.” In April, the measured LDL value “150” was high and exceeded the LDL reference range. Consequently, the converted LDL value “22.5” is high and exceeds the common reference range “0-10.” In fact the converted data value 140 for the April LDL reading was 22.5. It should be noted that when the acquired value reads below or above the original reference range, the converted value will similarly and proportionately read below 0 or above 10.

As demonstrated in FIG. 2 the patient's blood was tested for two purposes, namely cholesterol and LDL. While originally two different reference ranges were used to test the patient's health namely “160-200” for cholesterol and “60-100” for LDL, using the conversion method of the present invention one common reference range was used for both tests namely “0-10.”Therefore when the health care professionals evaluate a person's health, the health care professional need not worry about which reference range applies to which test. Instead one common reference range has been used for all tests involved.

FIG. 3a in conjunction with FIG. 3b depict the complexities of most blood chemistry reports. As shown in both figures over 25 aspects of the patient's blood have been tested over a three day period, February 13- February 15. Sodium (NA), potassium (K), and calcium (CL) represent just a portion of the aspects tested by the blood test of FIG. 3a-3b. FIG. 3a depicts a prior art blood chemistry report. In the far left column the various tests conducted are shown and in the far right column the reference ranges 130 for these tests are shown. In FIG. 3a, sodium, (NA) has a reference range 136-148, while potassium (K) has a reference range 3.5-5. A health care professional when evaluating the blood test results must carefully follow each line to prevent the wrong reference range from being applied against the wrong acquired value. The acquired values are depicted in the three middle columns, one column for each day.

FIG. 3b depicts the simplified version of the blood chemistry report of FIG. 3a in accordance with the method of the present invention. As depicted in FIG. 3b only one reference range “0-10” has been used for all 25 aspects of the patient's blood. By quickly reviewing the results shown in FIG. 3b, it becomes quickly apparent which of the patient's test results run high because those values run 10 and higher which of the results run low because those values run below 0. In FIG. 3b, a reference range column is unnecessary because one common reference range has been utilized. By eliminating the need for individual reference ranges, the present invention simplifies the lab report for the health care professional and consequently, reduces room for error.

FIGS. 4a-4b depict another prior art blood chemistry report and modified blood chemistry report pair. In FIG. 4a, once again a list of acquired values 120 with a corresponding list of reference ranges 130 to be used when evaluating the acquired values 120 is depicted. FIG. 4b represents the blood chemistry report of FIG. 4a as modified using the conversion method of the present invention. As shown in FIG. 4b all the acquired values of FIG. 4a have been converted proportionately to a scale of 0-10.

FIG. 5 depicts a further table demonstrating the conversion method of FIG. 1. In particular, FIG. 5 depicts a glucose reading. The glucose value was tested four times a day over a week long period. The third column in FIG. 5 represents the measured data values 120 in accordance with the original reference range “60-180” while the fourth column represents the measured data values as converted 140 to the common reference range “0-10.”

FIG. 6 depicts the conversion process of the present invention in accordance with a second illustrative embodiment, medication. The conversion process of the present invention could easily be used to prevent overdose. FIG. 6 depicts the dosage range for the medication Isordil “2.5 mg-160 mg” and Plendril “1.25 mg-10 mg.” As can be seen in FIG. 6, a patient can withstand larger doses of Isordil than Plendril. By way of example should a patient receive 40 mg of Plendril, the patient would overdose, whereas according to the chart of FIG. 6 should the patient receive 40 mg of Isordil the patient would be within an acceptable reference range. By converting the dosage ranges or the acquired values 120 for Isordril and Plendril to a common medication reference range 140 the risk of overdose in minimized. In the medication dusbursement embodiment, the common reference range “0-10” ranges from “0” the placebo to “10” the maximum dosage.

While fundamental features of the invention have been shown, described, and pointed out, as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and steps that perform substantially the same function, in substantially the same way, bearing substantially the same results are within the scope of the invention. Substitution of elements from one described embodiment to another are also solely intended and contemplated. It is also to be understood that the drawings are not necessarily drawn to scale, but they are merely conceptual in nature. It is the intention, therefore, to be limited only as indicated by the scope of the claims appended hereto.

I claim:

1. A method for converting data from a first reference range particular to a medical test to data related to a universal reference range common to a plurality of medical tests, comprising the steps of:
acquiring data from a test, the test having a first reference range, the first reference range particular to the test, the first reference range having a low value and a high value, the acquired data proportionately related to the first reference range, the proportionate relationship of the acquired data to the first reference range indicative a health condition; and
converting the acquired data to a second reference range, the second reference range universal to a plurality of tests, the second reference range having a low value and a high value, the converted data proportionately related to the second reference range,
wherein the proportionate relationship of the converted data to the second reference range substantially parallels the proportionate relationship of the acquired data to the first reference range, and
wherein the proportionate relationship of the converted data to the second reference range indicates the same health condition as the proportionate relationship of the acquired data to the first reference range.

2. A method as in claim 1, wherein the converting step converts using a first formula:
X=[(M−R1)/(R2−R1)]10
wherein “X” represents the converted data value; “M” represents the acquired data value; “R1” represents the low value of the first reference range; and “R2” represents the high value of the first reference range.

3. A method as in claim 1, wherein each test of the plurality of tests indicates a different health condition.

4. A method as in claim 1, wherein the low value of the second reference range is at least one of zero and one.

5. A method as in claim 1, wherein the high value of the second reference range is at least one of ten, one hundred and one thousand.

6. A method for converting drug prescription to a universal reference range common to a plurality of drug prescriptions, comprising the steps of:
acquiring a prescribed dosage amount for a drug, the prescribed dosage amount proportionately related to a first reference range particular to the prescribed drug, the first reference range having a minimum dosage amount and a maximum dosage amount;
converting the prescribed dosage amount to a second reference range, the second reference range universal to a plurality of drugs, the second reference range having a minimum dosage amount and a maximum dosage amount, wherein a proportionate relationship of the converted dosage amount to the second reference range substantially equals the proportionate relationship of the acquired dosage amount to the first reference range.

7. A method as in claim 6, wherein the converting step converts using a first formula:
X=[(M−R1)/(R2−R1)]10
wherein “X” represents the converted dosage amount; “M” represents the prescribed dosage amount; “R1” represents the minimum dosage amount of the first reference range; and “R2” represents the maximum dosage amount of the first reference range.

8. A method as in claim 6, wherein the low value of the second reference range is at least one of zero and one.

9. A method as in claim 6, wherein the high value of the second reference rance is at least one of ten, one hundred and one thousand.

10. A method for converting data from a first reference range particular to a medical test to data related to a universal reference range common to a plurality of medical tests, comprising the steps of:
acquiring data from a test, the test having a first reference range, the first reference range particular to the test, the first reference range having a low value and a high value, the acquired data proportionately related to the first reference range, the proportionate relationship of the acquired data to the first reference range indicative a health condition; and
converting the acquired data to a second reference range, the second reference range universal to a plurality of tests, the second reference range having a low value and a high value, the converted data proportionately related to the second reference range,
wherein the proportionate relationship of the converted data to the second reference range substantially parallels the proportionate relationship of the acquired data to the first reference range,
wherein the proportionate relationship of the converted data to the second reference range indicates the same health condition as the proportionate relationship of the acquired data to the first reference range, and
wherein the converting step converts using a first formula:
X=[(M−R1)/(R2−R1)]10
wherein “X” represents the converted data value; “M” represents the acquired data value; “R1” represents the low value of the first reference range; and “R2” represents the high value of the first reference range.
